(19) United States
(12) Patent Application Publication
(10) Pub. No.: US 2022/0403364 A1
(43) Pub. Date: Dec. 22, 2022

(54) BACTERIA IN 3D POROUS MEDIA

(71) Applicant: The Trustees of Princeton University, Princeton, NJ (US)

(72) Inventors: Sujit Datta, Princeton, NJ (US); Tapomoy Bhattacharjee, West Bengal (IN)

(73) Assignee: The Trustees of Princeton University, Princeton, NJ (US)

(21) Appl. No.: 17/608,018

(22) PCT Filed: Apr. 28, 2020
(86) PCT No.: PCT/US2020/030213

§ 371 Date: Nov. 1, 2021

Related U.S. Application Data
(60) Provisional application No. 62/841,334, filed on May 1, 2019.
(60) Provisional application No. 62/890,212, filed on Aug. 22, 2019.

(51) Int. Cl. C12N 11/04; C12N 1/04; C12M 3/06; C12M 1/12; C12M 1/34; C12N 1/20

(52) U.S. Cl. CPC C12N 11/04; C12N 1/04; C12M 23/16; C12M 25/14; C12M 41/46; C12N 1/20; C12N 2533/54; C12N 2533/70

(57) ABSTRACT

Disclosed is a 3D porous medium and a method of manufacture. The 3D porous medium includes (i) a support structure of transparent hydrogel particles or emulsion droplets, (ii) bacterial nutrient in open volumes between the transparent hydrogel particles, as well as within micropores in the transparent hydrogel particles, and (iii) bacterial cells within the open volumes in the support structure.

### CROSS-REFERENCE TO RELATED APPLICATIONS

This application claims priority to U.S. Provisional Patent Application Nos. 62/841,334, filed May 1, 2019, and 62/890,212, filed Aug. 22, 2019, and which are hereby incorporated in their entirety by reference.

### TECHNOLOGICAL FIELD

This application is drawn to 3D porous medium, and particularly to 3D porous medium that allow targeted placement of bacteria within the 3D porous me chum.

### BACKGROUND

While bacterial motility is well-studied in unconfined liquid media and at flat surfaces, in most real-world settings, bacteria must navigate heterogeneous 3D porous media. For example, during an infection, pathogens squeeze through pores in tissues and biological gels, enabling them to spread through the body. This process can also be beneficial; for example, a promising route toward cancer treatment relies on engineered bacteria penetrating into tumors and delivering anticancer agents. In agriculture, the migration of rhizosphere bacteria through soil impacts crop growth and productivity, while in environmental settings, the process of bioremediation relies on motile bacteria migrating towards and degrading contaminants trapped in soils, sediments, and subsurface formations.

However, despite their potentially harmful or beneficial consequences, how motile bacteria move through 3D porous media remains completely unknown. As a result, our ability to accurately model migration in porous media is limited. This gap in knowledge hinders attempts to model the spread of infections, predict and control bacterial therapies, and develop effective agricultural and bioremediation strategies.

In free solution, peritrichous bacteria are propelled by a rotating bundle of flagella along ballistic runs of mean speed (vᵣ) and length (Lᵣ); these are punctuated by rapid tumbles, arising when flagella spontaneously unbundle, that randomly reorient the cells. This run-and-tumble motion is thus diffusive over time scales larger than the run duration, with a translational diffusivity given by vᵣLᵣ/3. How this behavior changes in a porous medium is unclear. A common assumption is that the bacteria continue to perform runs with a mean run speed (vᵣ), but with a shorter length (L′ᵣ<Lᵣ) due to collisions with the solid matrix of the medium. These are thought to reorient the cells in a manner similar to tumbles, leading to a decreased diffusivity vᵣL′ᵣ/3. However, how to determine L′ᵣ is unclear, and in practice it is approximated by the average pore size or acts as an ad hoc parameter. Thus, while this approach is appealing due to its simplicity, it provides little fundamental understanding of how bacteria migrate in porous media. Indeed, the underlying assumption that the cells move via run-and-tumble motility has never been verified; typical 3D porous media are opaque, precluding direct observation of bacterial motion in the pore space.

Thus, what is needed is a 3D porous media that allows direct visualization, at single-cell resolution.

### BRIEF SUMMARY

The disclosure is generally drawn to 3D porous media containing bacteria, and systems and methods for visualizing bacteria within a 3D porous medium. In particular, systems and methods that allows bacteria to be inserted into the 3D porous medium in a controlled fashion (e.g., at discrete locations, at certain concentrations, etc.), as well as the resulting 3D porous medium containing the bacteria.

A first aspect of the present invention is drawn to a 3D porous medium that includes three components: (i) a jammed support structure comprising a plurality of transparent hydrogel particles or emulsion droplets; (ii) a bacterial nutrient of a desired composition in the open volumes between transparent hydrogel particles or emulsion droplets and within micropores in the transparent hydrogel particles (if hydrogel particles are used); and (iii) a plurality of bacterial cells within at least some of the open volume formed between transparent hydrogel particles.

Optionally, the transparent hydrogel particles may have a mass fraction between 0.5%-2.5% of the combined mass of the plurality of transparent hydrogel particles and the bacterial nutrient. The internal mesh size of the jammed support structure may be between 5 nm and 200 nm. The hydrogel particles may have a diameter between 10 nm and 100 μm.

Optionally, the 3D porous medium may contain multiple strains of bacteria. For example, with environmental samples contain a mixture of many strains of bacteria from soil, sediment, lungs, guts, other human and animal tissues, etc. In some cases, one or more locations contains a mixture of strains, while in other cases, each strain is present at a different location within the porous medium. In some cases, some or all of the bacterial cells include a marker gene.

In some cases, the 3D porous medium may consist, or consist essentially of, the plurality of transparent hydrogel particles, the plurality of bacterial cells, and the bacterial nutrient. In other cases, the 3D porous medium may also include other components. For example, the porous medium may optionally include a test mineral or chemical (including, e.g., non-metabolizable attractants and/or repellants, signaling molecules, antimicrobials, antibiotics, biological polymers, etc.) in the open volumes between transparent hydrogel particles and within micropores in the transparent hydrogel particles. Other components that may be included are polymers, that may be used, e.g., to modify the structural and/or rheological characteristics of the 3D porous medium.

Optionally, the 3D porous medium may have different compositions/concentrations of bacterial nutrients at different locations within the medium. For example, it may be beneficial to have an area of the medium that is nutrient-rich and one that is nutrient-poor, or have a nutrient in one area that is not present in another area. In some embodiments, the desired composition in some locations may include low concentrations as in starvation or minimal media (i.e., does not need to have nutrients appropriate for a particular bacteria species, just salts to maintain osmotic pressure). In some cases, the pH of the nutrients may be controlled, such as being less than, e.g., 7.2.

A second aspect of the present disclosure is drawn to a kit for creating 3D porous media. The kits may include hydrogel particles and a liquid or powder capable of being used by itself, or with the addition of DI water, to form a bacterial growth medium adapted for swelling the hydrogel particles.

A third aspect of the present disclosure is drawn to a method for producing a porous medium incorporating bacteria. The method first includes providing a porous medium, then using a nozzle to introduce a bacterial cell suspension to at least one location within the porous medium, typically at a controlled concentration, rate, pressure, etc. Each bacterial cell in the bacterial cell suspension may comprise a marker gene. Then, the nozzle is removed, and the porous medium is allowed to at least partially self-heal.

Optionally, the method includes additional steps, such as continuously introducing the bacterial cell suspension into the porous medium as the nozzle is moved within the porous medium from a first location to a second location, and/or allowing the bacterial cells to grow and move within the porous medium. Other optional steps include utilizing fluorescence microscopy (such as confocal microscopy) to visualize cell fluorescence in order to, e.g., characterize microbial motility or microbial growth under different conditions (e.g., different pore size, permeability, porosity, medium stiffness, chemical environment, flow conditions, etc.), or interacting with a bacterial cell or other mineral or chemical in the porous medium (including, e.g., removing or recovering cells from the porous medium for off-line assaying). This could be done, e.g., with a microcapillary or other type of micronozzle. Still other optional steps include introducing a test mineral or chemical to at least one location within the porous medium, and/or controlling fluid flow, nutrient signals, chemical signals, or a combination thereof throughout the porous medium using microfluidic channels.

Optionally, the porous medium may be provided by, e.g., swelling dry hydrogel granules in a bacterial cell culture media at a predetermined concentration. In some cases, at least one attribute of the porous medium (e.g., pore size, permeability, porosity, elastic modulus, viscous modulus, yield stress, etc.) is controlled using different hydrogel concentrations, wherein the attribute is selected from the group consisting of pore size, permeability, porosity, elastic modulus, viscous modulus, and yield stress.

### BRIEF DESCRIPTION OF THE DRAWINGS

FIG. 1 is a cross-sectional view of a 3D porous medium according to an embodiment of the present disclosure.

FIG. 2 is a cross-sectional view of a 3D porous medium according to an embodiment of the present disclosure.

FIG. 3A is a flowchart illustrating an embodiment of a method of the present disclosure.

FIGS. 3B, 3C, and 3D are black-and-white images of fluorescence images that illustrate different patterns that bacteria can be arranged in within a porous me chum.

FIG. 3E is a flowchart illustrating an embodiment of a method of the present disclosure.

FIG. 4A is a graph of a representative MSD of a tracer particle, used for measuring the length scale at which the tracer MSD plateaus, providing a measure of the smallest confining pore size of the medium.

FIG. 4B is a graph showing ensemble average mean squared displacement (MSD) as a function of lag time for unconfined bacteria 301 and for bacteria in porous media with increasing amounts of confinement (characteristic pore sizes of 3.6 μm 302, 2.5 μm 303, 1.9 μm 304, and 1.5 μm 305). The dark circles 311, 312, 313, 314, and 315 indicate deviation from ballistic motion for unconfined bacteria, or deviation from superdiffusive motion for bacteria in porous media.

FIG. 4C is a graph illustrating mean squared displacement of a single cell inside a porous medium with a characteristic pore size of 3.6 μm, showing that at long timescales the motion of individual cells becomes diffusive after transient subdiffusion.

FIG. 5A is a graph illustrating the probability density of measured hopping lengths Q(Lₕ) in porous media maintained at different temperatures, all with the same characteristic pore size.

FIG. 5B is a graph illustrating the probability density of measured trapping durations P(τₜ) in porous media maintained at different temperatures. The different measurements at different temperatures are offset vertically for clarity; each data set has its own shape-coded axis. The lines indicate power-law scaling for large τₜ, P(τₜ)˜τₜ^{-α}.

FIG. 5C is a graph illustrating the measured power-law exponent a decreasing with decreasing temperature, and thus with mean swimming speed, indicating a broader distribution of trapping durations.

FIG. 6A is a graph illustrating the impact of the impact of different levels of an antibiotic (here, kanamycin) on bacterial growth in the porous medium.

FIG. 6B is a graph illustrating the impact of the impact of different levels of an antibiotic (here, ampicillin) on bacterial growth in a traditional liquid growth medium.

FIG. 6C is a graph illustrating the impact of the impact of different levels of an antibiotic (here, ampicillin) on bacterial growth in the porous medium.

FIG. 7A is a graph illustrating the probability density of measured hopping lengths Q(Lₕ) in porous media of different characteristic pore sizes all maintained at 30° C. The solid lines indicate the measured chord length distribution Ξ(Lₕ) for each medium (from left to right at a probability density of 3×10⁻³, the lines are from the smallest to largest pore sizes—1.5 μm, 1.9 μm, 2.5 μm, and 3.6 μm). Both Q(Lₕ) and Ξ(Lₕ) narrow as the characteristic pore size is decreased, and reasonable agreement is found between the two.

FIG. 7B is a graph illustrating the probability density of measured trapping durations P(τₜ) in porous media maintained at different temperatures. The different measurements at different temperatures are offset vertically for clarity; each data set has its own shape-coded axis. The lines indicate power-law scaling for large τₜ, P(τₜ)˜τₜ^{-α}.

FIG. 7C is a graph illustrating the measured power-law exponent a decreasing with decreasing characteristic pore size, indicating a broader distribution of trapping durations.

### DETAILED DESCRIPTION

As used herein, articles such as “a” and “an” when used in a claim, are understood to mean one or more of what is claimed or described.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used here, the term “self healing” means that when a small value of shear stress (lower than the “yield stress”, defined below) is applied to the medium, the medium does not deform irreversibly and the pore size distribution medium recovers its initial distribution on the withdrawal of the shear stress, while when a shear stress larger than the yield stress is applied to the medium, the individual hydrogel particles rearrange with respect to each other and the medium becomes fluidized. This feature enables the use of a nozzle to inject bacteria and materials into the medium: by applying a local stress larger than a yield stress, an injection nozzle can move through the porous medium and extrude cells into the pore space, and after extrusion of cells into the pore space, the nozzle is moved away, and the surrounding hydrogel particles rapidly re-densify around the cells, re-forming the solid matrix of the medium. The yield stress is defined through the mechanical response of the medium to an applied shear rate: when a low value of shear rate is applied to the medium, the medium exhibits a constant shear stress (known as the “yield stress”) independent of the applied shear rate, while at sufficiently large shear rates, the shear stress increases with applied shear rate.

As used herein, the term “transparent hydrogel particles” means hydrogel particles that individually have a percent transmission of light in the visible light spectrum of greater than 50%, and preferably greater than 75%, and more preferably greater than 90%.

Microbial communities critically impact our everyday lives, with potentially harmful consequences such as causing disease and fouling industrial equipment. In other cases, microbes can have beneficial impacts, including protecting against pathogens, degrading contaminants, fixing nitrogen, and decomposing organic matter. In most of these cases, the cells inhabit heterogeneous three dimensional (3D) porous spaces such as gels, tissues, foods, and soils. By contrast, lab assays of cellular behavior typically study microbes in free liquid culture or on uniform flat surfaces. While these approaches yield key insights, growing evidence suggests that they do not adequately replicate the complexity of natural habitats: laboratory measurements of microbial behavior—e.g. of growth rates, metabolism, virulence, and antibiotic resistance—often strongly differ from measurements performed in real-world settings.

Metagenomics approaches provide a wealth of information on which microbes are present in natural settings. However, such bulk approaches mask heterogeneities in cellular behavior and community organization. Laboratory studies using liquid cultures in test tubes or microwell plates, isolated cells in droplets or particles, and communities grown on flat surfaces in Petri dishes shed additional light on microbial physiology. However, these approaches do not recapitulate the complexity inherent in natural porous habitats.

Inoculation of cells into soft “semisolid” agar is a common way of assaying microbial motility in a porous material. However, this approach is not generally used for other biological assays for three key reasons: there is limited control over the properties of the agar matrix, the turbidity of agar precludes visualization of individual cells, and it is challenging to non-destructively remove cells and cellular material for further assays. Recently-developed biofabrication approaches also provide the ability to arrange microbes in 3D. However, these approaches work by constraining cells within a cross-linked gel matrix, which hinders them from moving, mixing, and organizing as in their native habitats. Moreover, cross-linked gels eventually rupture due to cell overgrowth, precluding long-time studies. Finally, cross-linked gels must be irreversibly broken to retrieve cells and cellular material. By using a self-healing matrix of densely-packed hydrogel particles, the presently disclosed system and method overcomes all of these limitations.

The presently disclosed porous medium can function as a “porous petri dish” that enables studies of microbes in 3D porous habitats. Specifically, this platform enables microbial cells to be arranged—in any desired 3D structure, with any community composition—within a porous matrix having tunable properties. Crucially, the matrix enables microbial communities to be sustained over long times, visualized using fluorescence techniques, and profiled using a broad range of biological assays. Hence, the ability of this platform to study microbial communities in porous habitats has enormous research and applied potential, impacting the study and treatment of microbes in the gut, the lung, the skin, and in soil.

Initial investigations using the disclosed platform have already revealed previously-unknown differences in the motility and antibiotic susceptibility of microbes inhabiting porous matrices compared to those in liquid culture/on flat surfaces. For example, measurements of microbial spreading in the disclosed matrices differ from those performed in liquid cultures by a factor of ˜500.

The presently disclosed platform relies on embedding microbial cells within a self-healing support matrix. The matrix is comprised of densely-packed, biocompatible hydrogel particles swollen in liquid bacterial culture. This matrix is a soft solid; however, unlike with cross-linked gels used in current bioprinting approaches, an injection micronozzle can move freely inside the matrix along any prescribed 3D path by locally rearranging the hydrogel particles. These particles then rapidly re-densify and self-heal around injected microbes, supporting the cells in place without any additional harmful processing.

This approach is therefore analogous to 3D printing. For example, dilute cells can be dispersed within the support matrix, enabling studies of the motility of individual bacteria. 3D-printed communities can be created in structures composed of hundreds of thousands of cells. This approach also enables other compounds, e.g., nutrient sources, to be positioned throughout the matrix. The internal mesh size of the individual hydrogel particles is ˜100 nm, smaller than the individual cells, but large enough to allow unimpeded transport of metabolites, signaling molecules, and antibiotics. The pores between the particles can be tuned to different sizes, enabling nutrient and reagent delivery, waste removal, and cellular migration throughout. The hydrogel matrix can therefore support the growth of a microbial community over extended periods of time, at least >100 hours in some tests.

Moreover, because the support matrix is swollen in aqueous liquid, it is transparent. This feature enables full visualization of individual cell(s) up to entire communities expressing fluorescent proteins—e.g., reporting key cellular processes such as viability, growth, competence, quorum sensing, and virulence—at single-cell resolution via confocal microscopy. This includes visualizing the flagellar appendages of individual cells, and visualizing the distribution of multiple cells as they migrate through the medium. It also enables fluorescent probes to be introduced and visualized throughout the community without perturbing it, e.g., lectins for polysaccharide content in biofilms, nucleic acid probes to identify specific species via fluorescence in situ hybridization, and sensors of oxygen levels. Hence, this technology presents a unique opportunity to interrogate microbial behavior in porous habitats.

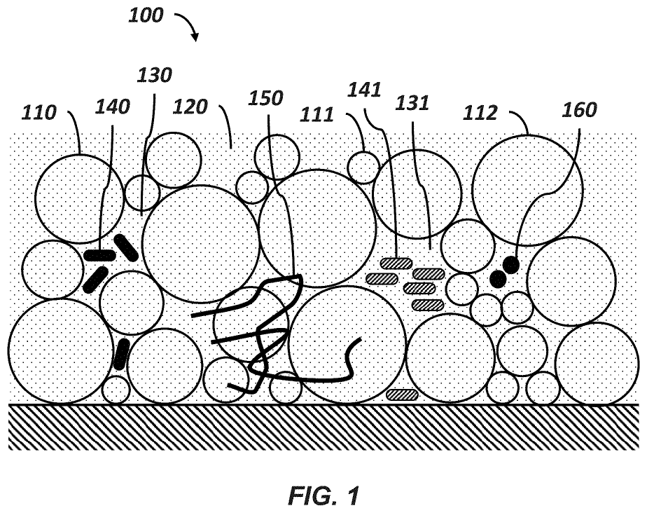

A first aspect of the present disclosure is drawn to a 3D porous medium. This can best be understood with respect to FIG. 1. As seen in FIG. 1, the synthetic 3D porous medium 100 will include a plurality of transparent hydrogel particles 110, 111, 112 that form a jammed support structure.

The transparent hydrogel particles may be comprised of any appropriate material. Preferred embodiments utilize synthetic materials. In some embodiments, this may include a crosslinked polyethylene glycol, a crosslinked polyacrylamide, a crosslinked polyvinyl alcohol, crosslinked dextran, crosslinked agarose, crosslinked poly n-isopropylacrylamide, Matrigel® extracellular matrix, crosslinked collagen, crosslinked fibrin, crosslinked hyaluronic acid, crosslinked alginate, crosslinked gelatin, and/or laponite. In some embodiments, this may include a carbomer. Carbomers are synthetic high-molecular-weight polyacrylic acids that are typically cross-linked with allyl sucrose or allyl pentaerythritol and typically contain between 56 and 68% w/w carboxylic acid groups. The molecular weight of carbomer is sometimes estimated to be at 7×10⁵ to 4×10⁹ Da. As three-dimensionally cross-linked hydrogels, carbomers do not dissolve but can swell to a remarkable extent in water after neutralization to form a hydrogel. A preferred embodiment uses randomly crosslinked acrylic acid/alkyl acrylate copolymers, such as Carbomer 980 sold by Ashland.

The 3D porous medium 100 will also include a bacterial nutrient 120 in the open volumes 130, 131 between transparent hydrogel particles and within micropores (not shown) in the transparent hydrogel particles. The bacterial nutrient 120 will typically be a liquid solution containing nutrients appropriate for the type of bacteria the nutrients are intended to support.

In some embodiments, the hydrogel is formed by swelling dry hydrogel granules in a bacterial cell culture media. That culture media could then function as the bacterial nutrient 120. In some cases, the mass fraction of the plurality of transparent hydrogel particles is between 0.5%-2.5% of the combined mass of the plurality of transparent hydrogel particles and the bacterial nutrient. As an example, 1.5 grams of dry granules of Carbomer 980 can be dispersed directly into 98.5 grams of bacterial nutrient—a liquid media comprising 2 wt % of a lysogeny broth powder in DI water—and mixing the suspension for an appropriate period of time, which in some embodiments is 10 minutes or more, 1 hour or more, or 12 hours or more, and may be 15 minutes or less, 2 hours or less, or 24 hours or less, or any combination thereof. For example, in some embodiments, the mixing time is between 10 and 15 minutes. In others, it is between 1 hours and 2 hours, while in others, it is between 2 hours and 12 hours. Any known broth or broth powder could be used; for example, in some embodiments, a marine broth is used. In some embodiments, between 0.5% and 2% mass fraction, such as between 0.5% and 1% mass fraction, of hydrogel particles are used in a Lysogeny Broth (Lennox), Lysogeny Broth (miller), EZ Rich, or Terrific Broth (TB) media. In some embodiments, between 0.75% and 2.5% mass fraction of hydrogel particles are used with M9 minimal media. After the particles swell, the pH can then be adjusted as desired. Taking the previous example with Carbomer 980, the pH can be adjusted to 7.4 by adding a basic material such as NaOH as appropriate. In some cases, the pH is adjusted to a final pH of about 7.4 or less, about 7.2 or less, or about 7.0 or less. In some embodiments, the pH is less than about 7.2. This protocol will result in a jammed, solid matrix or support structure of dense-packed hydrogel particles.

In some embodiments, the internal mesh size of the jammed support structure is between 5 nm and 200 nm. In some embodiments, the average particle size diameter of the transparent hydrogel particles is between 10 nm and 100 μm. In some embodiments, each of the transparent hydrogel particles has a diameter between 10 nm and 100 μm.

In some embodiments, the mechanical properties of the porous medium can then be characterized using rheology to determine if the media will exert significant mechanical stresses on the bacteria. For example, if the porous medium was determined to have a linear shear modulus and a bulk modulus more than 100 times the bacterial cell wall stiffness (˜100 MPa), one would not expect that the media would exert significant mechanical stresses on the bacteria.

In some embodiments, the range of physico-chemical conditions that can be tested using the disclosed platform can be expanded by fabricating hydrogel particles of different sizes (e.g., from 10 nm-100 μm) and different chemical functionalities (e.g., surface charge and chemistry, as is understood by those of skill in the art).

As seen in FIG. 1, when the hydrogel forms open volumes 130, 131 between the swollen hydrogel granules. Each individual swollen hydrogel granule will likely also include micropores (not shown). The bacterial nutrient will typically be present in every open volume of the a jammed support structure and in all micropores of the hydrogel particles.

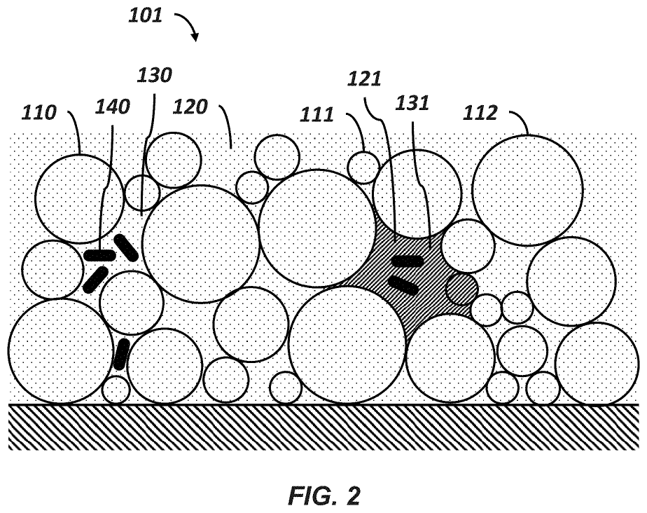

Referring briefly to FIG. 2, in some embodiments, the bacterial nutrient 120, 121 in the 3D porous medium 101 may have different compositions in different locations. Specifically, a composition of the bacterial nutrient 120 in a one of the open volumes 130 may be different from a composition of the bacterial nutrient 121 in a different open volume 131 of the jammed support structure.

It is noted that in some embodiments, a jammed support structure may also be created using emulsion droplets, rather than hydrogel particles. The overall arrangement and configuration is substantially the same as that shown in FIG. 1, but as understood in the art, this require an emulsion in which the volume fraction of the dispersed phase is sufficiently large that the emulsion is jammed (i.e., is solid-like with an elastic shear modulus G′>viscous shear modulus G″). Two ways of accomplishing this is (i) by using an emulsion in which the dispersed phase is above random close packing, and (ii) by forming an emulsion gel. Schematically, looking at FIG. 1, the spherical hydrogel particles 110, 111, and 112 would be replaced by emulsion droplets having the same shape(s), locations, and sizes.

The minimum amount of surfactant needed would be the amount needed to coat the dispersed phase droplet surfaces. As is understood in the art, this is partially based on particle size, and typically corresponds to surfactant concentrations ranging from 0.1 mM to 10 mM. Whether the surfactant interferes with bacteria growth and/or movement will depend on the application; typically, surfactants have dimensions in the nm range, three orders of magnitude smaller than the cell body size. Moreover, surfactants often help to lubricate interfaces, making them “slippery”. So, the use of an emulsion may be preferable to hydrogels in some applications.

Some embodiments that utilize an emulsion droplet may include, but is not limited to: (i) a dispersed phase that comprises or consists of a silicone oil and/or a fluorocarbon oil; (ii) a continuous phase that comprises or consists of a cell culture media (e.g., the same cell culture media as described previously) with, e.g., sufficient glycerol added for refractive index matching between the dispersed and continuous phase; and (iii) a surfactant the comprises or consists of sodium dodecyl sulfate, and/or surfactants in the Pluronic®, Tween®, Span®, and Brij® families of surfactants and detergents. For example, one such emulsion utilizes a 65% or greater volume fraction (such as between 0.68 and 0.73) of silicone oil droplets dispersed in LB media with 50%, where the droplets are sterically stabilized by Pluronic P105, a non-ionic amphiphilic copolymer.

When using emulsion droplets, the pore size of the medium would still be determined by how jammed it is, i.e., by the volume fraction of the dispersed phase, as well as by the sizes of the emulsion droplets and whether or not there are any attractive interactions between them (which would be tuned by surfactant). Typical Pore sizes that can be achieved using this method are between ˜10 nm and ˜10 micrometers.

Referring again to FIG. 1, the 3D porous medium 100 will also include a plurality of bacterial cells 140, 141 within at least some of the open volume 130, 131 formed between transparent hydrogel particles. The bacterial cells may include one or more strains. Any appropriate strain can be used. In some embodiments the strains comprise or consist of, E. coli, P. putida, V. cholerae, P. aeruginosa, and/or B. subtilis. In some embodiments, multiple strains will be present within the porous medium, including a first strain 140 and a second strain 141. For example, with environmental samples contain a mixture of many strains of bacteria from, e.g., soil, sediment, lungs, guts, other human and animal tissues, etc. In some embodiments, different strains are present at different locations within the porous medium. As shown in FIG. 1, in some embodiments, some or all of the strains are not present in the same open volume (here, only strain 140 is present in void 130, and only strain 141 is present in void 141). However, in other embodiments, some or all of the strains may be present in an open volume with at least one other strain—that is, in some cases, strain 140 and 141 could be present in void 130. In some embodiments, each strain is present at a different location within the porous medium.

In some embodiments, the bacterial cells include wild-type strains of bacteria and/or strains that have been modified to include one or more screenable marker genes. Screenable marker genes include, e.g., those that encode for the expression of a fluorescent protein such as sfGFP. In some embodiments, the bacterial cells consist only of strains that include a marker gene—that is, all bacterial cells comprise a marker gene. In some embodiments, different strains of bacteria have different marker genes. For example, in one embodiment, each strain uses genes that encode for a different color fluorescent protein. In that example, a camera positioned above the porous medium would be able to distinguish between the strains, even if the bacteria occupy the same x-y coordinates in the 3D porous medium (e.g., if a first bacterium was located on a first “level” of the porous medium at (x,y,z1) and a second bacterium was located on a second “level” of the porous medium at (x,y,z2)), or if the bacteria colonies overlap or occupy at least some of the same general x-y-z coordinates (e.g., if a single pore contained multiple species of bacteria).

In some embodiments, such as that shown in FIG. 2, the 3D porous medium consists of, or consists essentially of, the plurality of transparent hydrogel particles, the plurality of bacterial cells, and the bacterial nutrient. When consisting essentially of those components, no additional component is included that will substantially impact the transparency of the hydrogel particles, the ability of the 3D porous medium to self-heal, or the ability for bacteria to grow and survive in the 3D porous medium.

However, in other embodiments, the 3D porous medium may include other optional components.

Referring back to FIG. 1, the 3D porous medium may optionally also include at least one polymer 150. Such polymers may include, e.g., any known polymeric rheological modifier. Such polymers may include, but are not limited to, a carbomer, polyethylene glycol, polyacrylamide, dextran, agarose, poly n-isopropylacrylamide, and/or alginate. Such polymers, if present, are typically present in a total amount less than about 10% by weight of the total mass of the 3D porous medium. Depending on, e.g., the degree of cross-linking in the polymer, the concentration of the polymer should be limited to levels that still allow at least some self-healing of the hydrogel.

The 3D porous medium may also optionally include a test mineral or chemical 160 in the open volumes between transparent hydrogel particles and/or within micropores in the transparent hydrogel particles. The test mineral or chemical 160 is typically a mineral or chemical species that is not a nutrient. For example, the test mineral or chemical could be non-metabolizable attractants and/or repellants, signaling molecules, antimicrobials, antibiotics, biological polymers, etc. Often, these test minerals or chemicals are added in order to determine the impact that test mineral or chemical has on a property or characteristic of some or all of the bacteria, such as bacterial motility, growth rate, etc.

A second aspect of the present disclosure is drawn to a kit for creating 3D porous media. The kit generally comprises hydrogel particles, as discussed previously, and also contains a liquid bacterial growth medium adapted for swelling the hydrogel particles. Thus, a kit may comprise Carbomer 980, and an appropriate amount of a lysogeny broth powder such that when DI water is added it can form a liquid media comprising 2 wt % of a lysogeny broth powder in DI water. Instructions may also be optionally included in the kit.

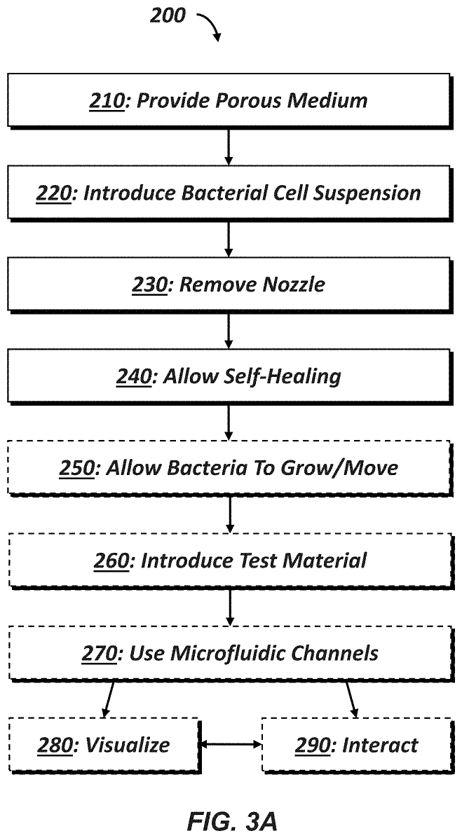

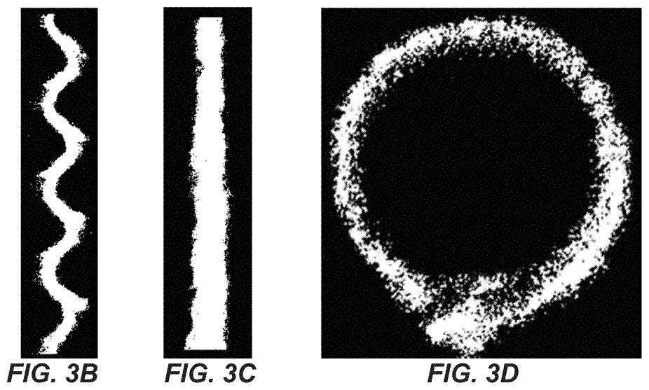

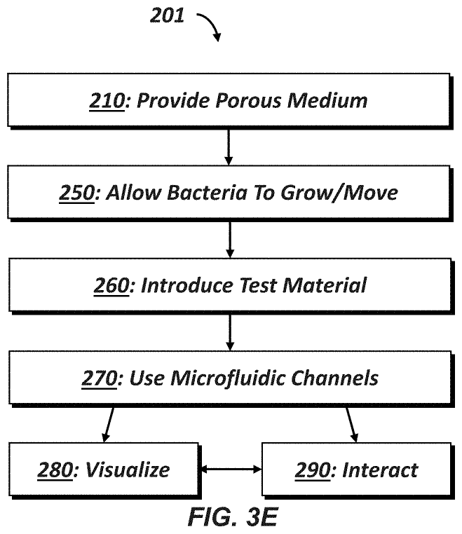

A third aspect of the present disclosure is drawn to a method for producing a porous medium incorporating bacteria. As seen in FIG. 3A, the method 200 generally involves providing a porous medium 210, i.e., the 3D porous medium disclosed previously, but without the bacterial cells present. In some embodiments, providing a porous medium comprises swelling dry hydrogel granules in a bacterial cell culture media at a predetermined concentration, as described previously. In some embodiments, at least one attribute of the porous medium is controlled or controllable based on hydrogel concentrations (e.g., rather than uniformly dispersing the hydrogel in a liquid media, modifying or controlling properties by using different concentrations of the hydrogel in different locations), wherein the attribute is selected from the group consisting of pore size, permeability, porosity, elastic modulus, viscous modulus, yield stress, etc.

Then, a nozzle is used to introduce 220 a bacterial cell suspension to at least one location within the porous medium, where each bacterial cell in the bacterial cell suspension comprises a marker gene.

The bacterial cell suspension may be any suspension that does not kill the bacterial cells, while allowing the bacterial cells to be injected or deposited into the porous medium. For example, it may be liquid bacterial cell culture media. The nozzle can be any appropriate nozzle connected to a reservoir of the bacterial cell suspension—a pipette, an injection micronozzle mounted to a motorized stage, the needle tip of a syringe, a nozzle from a 3D printer, etc.

Optionally, the nozzle may be moved to different locations within the porous medium (e.g., from a first location to a second location, whether in a straight line, in a pattern, etc.). In some cases, the nozzle may eventually return to the original location. In some cases, bacterial cell suspension is introduced into the porous medium only at discrete locations. In some cases, the nozzle is stopped when the bacterial cell suspension is introduced. In other cases, the bacterial cell suspension may be continuously introduced as nozzle moves from within the porous medium.

In some embodiments, the bacteria are arranged in an arbitrary manner. In other embodiments, the bacteria are arranged in various patterns, including but not limited to periodic waves (e.g., FIG. 3B), straight lines (e.g., FIG. 3C), and/or geometric shapes, including circles (e.g., FIG. 3D), or more complex arrangements.

The nozzle may then be removed 230 from the porous medium, and the porous medium is allowed to at least partially self-heal 240 after being disturbed by the nozzle.

Although additional, optional steps are shown in FIG. 3A as occurring in a sequential fashion following the self-healing of the porous medium, the following optional steps can be performed in any order, and may occur at earlier times in the process, as appropriate.

After the bacterial cell suspension has been introduced to the porous medium, the bacteria that was introduced may optionally be allowed to grow or move 250 within the porous medium for a period of time. In some embodiments, this period of time is at least 1 hour. In some embodiments, the period of time is between 1 hour and 24 hours.

After the porous medium has been provided, the porous medium can optionally be modified by introducing 260 a test mineral or chemical, as described previously, to at least one location within the porous medium. In some cases, different materials and/or different concentrations are used in different locations.

After the porous medium has been provided, the method 200 may optionally include controlling 270 fluid flow, nutrient signals, and/or chemical signals throughout the porous medium, by using microfluidic channels. For example, a test chamber can be provided for containing the 3D porous medium. The test chamber may have an array of microfluidic channels at its boundaries, enabling waste byproducts to be removed, nutrients/reagents to be controllably delivered, and fluid flow to be imposed as desired.

After the bacteria have been introduced to the porous medium, the method may optionally include utilizing some form of fluorescence microscopy, e.g., confocal microscopy, to visualize 280 cell fluorescence, for various purposes, including characterizing microbial motility and/or growth under different conditions, such as different pore size, permeability, porosity, medium stiffness, chemical environment, and/or flow conditions.

These tests may be useful for determining the true efficacy of a particular compound in a porous medium. For example, using the disclosed 3D porous medium, it can be seen that the Minimum Inhibitory Concentration (MIC)—the amount of antibiotics needed to suppress bacteria growth—inside a liquid culture (e.g., what is typically used to test antibiotics) can differ by a factor of ˜10 or more from the amount needed to suppress growth inside porous media.

After the bacteria have been introduced to the porous medium, the method may optionally include interacting 290 with the cell population, such as a bacterial cell or extracellular material in the porous medium. This may include, e.g., removing or recovering cells for off-line assaying, or moving one or more cells from one location to another. If a test mineral or chemical has been introduced 260, then the interaction 290 could also be with the test mineral or chemical. For example, the interaction could include removing or recovering chemicals such as metabolites, signaling molecules, and other chemicals that were produced by the bacteria, formed via a chemical reaction, or were added to the porous medium. Such interactions could be done with any appropriate means, including, e.g., a microcapillary or other type of micronozzle (e.g., syringe needle, pipette tip, etc.). Recovered cells or material can then undergo further testing. For example, in some embodiments, the recovered cells can be used to profile transcriptional changes by RNA-sequencing and the recovered extracellular material to profile small molecule and metabolite levels by mass spectrometry.

Referring briefly to FIG. 3E, it can be seen that a nozzle is not always required to provide the bacteria to the porous medium. In an alternate method 201, the process may skip some steps, if a random dispersal of bacteria within the porous medium is acceptable. In such cases, the cells can be simply be gently mixed (keeping shear stress above the yield stress) into the medium.

### EXAMPLE 1

In order to directly visualize bacterial motion in 3D porous media a 3D porous media was prepared by confining jammed packings of ˜10 μm-diameter hydrogel particles, swollen in liquid Lysogeny Broth (LB), in sealed chambers.

To prepare the jammed hydrogel porous media, dry granules of randomly crosslinked acrylic acid/alkyl acrylate copolymers (Carbomer 980, Ashland) were dispersed directly into liquid LB media (2 wt % of Lennox Lysogeny Broth powder in DI water). To ensure a homogeneous dispersion, the suspension was mixed for at least 12 hours. Since the hydrogel is a cross-linked network of negatively charged polyelectrolytes, the pH was adjusted to 7.4 by adding 10N NaOH. This protocol resulted in a jammed, solid matrix of dense-packed hydrogel particles.

The mechanical properties were characterized using rheology, and measure a linear shear modulus between 3 and 140 Pa for the packings. This modulus is ˜10⁶ times lower than the bacterial cell wall stiffness (˜100 MPa), and the corresponding bulk modulus is ˜10³ times lower than the cell wall stiffness; we therefore do not expect that the media exert significant mechanical stresses on the bacteria.

The internal mesh size of each particle was ˜100 nm—much smaller than the individual bacteria would be, but large enough to allow unimpeded transport of nutrients and oxygen. packings therefore act as solid matrices with macroscopic interparticle pores that bacteria can swim through. Importantly, because the hydrogel particles are highly swollen, light scattering from their surfaces is minimal. This porous media is therefore transparent, enabling direct visualization of bacterial motility in the 3D pore space via confocal microscopy.

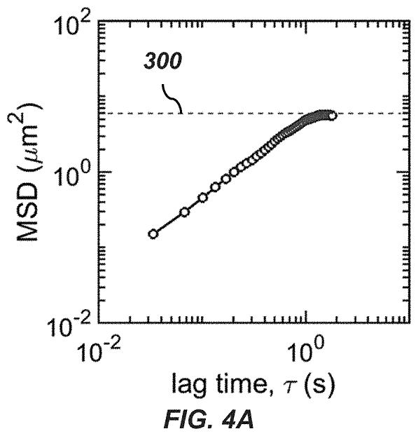

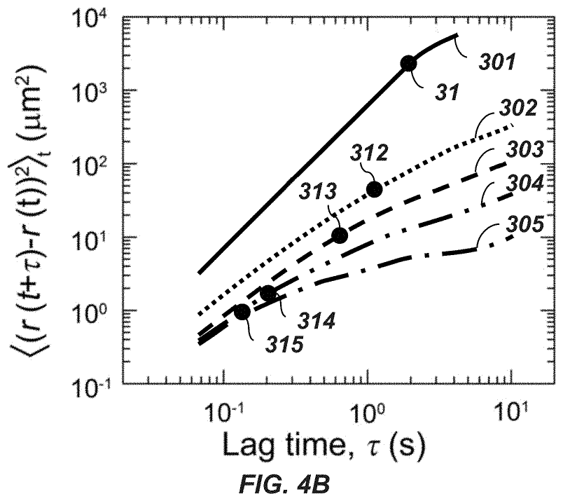

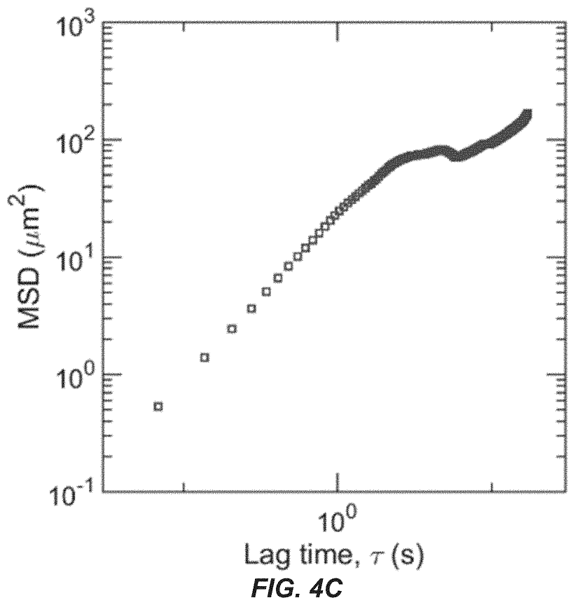

To tune the degree of pore confinement, we prepare four different media using different hydrogel particle packing densities. To create the different densities, different mass fractions of dry hydrogel granules were used—0.50%, 0.65%, 0.75%, and 0.85%. The pore space structure was characterized by dispersing 2×10⁻³ wt % of 200 nm carboxylated polystyrene fluorescent nanoparticles (FluoSpheres, Invitrogen), which have a zeta potential (approximately −20 mV) comparable to those of E. coli (approximately −30 mV), and tracking them. Because the tracers are larger than the hydrogel mesh size, but are smaller than the inter-particle pores, they migrate through the pore space. An in-house custom MATLAB script was used to track the individual particles, identifying each tracer center using a peak finding function with subpixel precision and tracking its motion using the classic Crocker-Grier algorithm. For each tracer, its mean-squared displacement (MSD) was calculated as a function of lag time. For short lag times, the tracer diffuses unimpeded, and the MSD varies linearly in time. At longer times, the tracer becomes constrained by the surrounding solid matrix, and the MSD plateaus (see dashed line 300 in FIG. 4A). To calculate the smallest confining pore size, take the square root of this plateau value and add the tracer particle diameter.

One can plot 1-CDF(a), where

$$CDF ( a ) = \frac{{\sum }_{0}^{a} a ρ ( a )}{{\sum }_{0}^{∞} a ρ ( a )}$$

is the cumulative distribution function of measured pore sizes and ρ(a) is the number fraction of pores having size a. In this example, increasing the concentration of dry hydrogel particles used to prepare the porous medium increases the density of the medium. Tuning the hydrogel particle packing density can provide a way to tune the pore size distribution, with, e.g., pores between 1 μm and 13 μm in the least dense medium (0.50% dry hydrogel granules), 1 μm to 10 μm with 0.65% dry hydrogel granules, 1 μm to 8 μm with 0.75% dry hydrogel granules, and 1 μm to 4 μm in the densest medium (0.85% dry hydrogel granules). The pore sizes follow an exponential distribution for all four media, indicating a characteristic pore size; for simplicity, each medium can be referred to by this characteristic size. These hydrogel packings therefore can serve as a model for many bacterial habitats, such as gels, soils, and sediments, which have heterogeneous pores ranging from ˜1 to 10 μm in size, smaller than the mean bacterium run length and for many pores, smaller than the overall flagellum length ˜7 μm.

An overnight culture of E. coli (W3110) was prepared that constitutively expresses green fluorescent protein (GFP) throughout the cytoplasm at 30° C. A 1% solution of this culture in fresh LB was incubated for 3 hours. At this point, the optical density is approximately 0.6. A small volume of this 0.6 OD culture was gently mixed into the hydrogel porous media to achieve a final bacterial concentration of 8000 cells/μL. This concentration is sufficiently dilute to minimize intercellular interactions, local gradients in oxygen or nutrient content, and changes in the overall concentration of oxygen and nutrients throughout the media; changes in cellular motility were not detected over the experimental time scale (˜30 min), in agreement with this expectation. As a negative control, a strain was tested that contained a deletion of the flagellar regulatory gene flhDC, which does not assemble flagella; negligible motility was detected for this strain, indicating that the results probe motility due to flagellar bundling and rotation. For another experiment, flagella were stained using Alexa Fluor dye, washing away free dye before mixing the bacterial culture with the hydrogel porous medium.

In unconfined liquid, E. coli exhibit run-and-tumble motility. To quantify this behavior, the center \vec{r}(t) of each individual cell was tracked with a time resolution of δt=69 ms, projected in two dimensions, and analyze the time- and ensemble-averaged MSD, (r(t+τ)−r(t))², as a function of lag time. For short lag times, the MSD varies quadratically in time, indicating ballistic motion due to runs with a mean speed vᵣ=28 μm/s. By contrast, above a crossover time of ≈2 s, which corresponds to the mean run duration, the MSD varies linearly in time. This transition to diffusive motion is consistent with previous measurements.

The influence of pore confinement on bacterial motion can also be investigated. The E. coli can be dispersed within the porous media at 6×10⁻⁴ vol %, sufficiently dilute to minimize nutrient consumption and intercellular interactions. Cell motion can be tracked for at least 10 s, five times larger than the unconfined run duration, and a subsequent analysis can focus on cells that exhibit motility within the tracking time. A mutant that cannot assemble flagella shows negligible motility, indicating that motion due to thermal diffusion and surface pili is insignificant. If pore confinement were to simply reduce the run length, as is often assumed, the MSDs would still exhibit a crossover between ballistic and diffusive motion, but at earlier lag times. Markedly different behavior from this prediction is found. FIG. 4B shows ensemble average mean squared displacement (MSD) as a function of lag time for unconfined bacteria 301 and for bacteria in porous media with increasing amounts of confinement (characteristic pore sizes of 3.6 μm 302, 2.5 μm 303, 1.9 μm 304, and 1.5 μm 305). The dark circles 311, 312, 313, 314, and 315 indicate deviation from ballistic motion for unconfined bacteria, or deviation from superdiffusive motion for bacteria in porous media. For short lag times, the MSDs vary as τ^{1.5}, indicating superdiffusive motion. By contrast, above a crossover time τ_{c} (dark circles 311, 312, 313, 314, and 315 in FIG. 4B), the MSDs vary as τ^{v}, where the exponent 0<v≤1 indicates subdiffusive behavior. Resealing each MSD by its crossover point can highlight these two regimes; moreover, it reveals that decreases with increasing pore confinement, approaching 0.5 for the densest medium. Analysis of the distribution of cell displacements at different lag times supports this finding. Note that close inspection of the individual cell MSDs reveals that the subdiffusion is transient: at sufficiently long lag times, individual MSDs can again become diffusive (See FIG. 4C), an effect that is masked in averaging. Such transient subdiffusion arises from transient trapping within a heterogeneous environment.

To monitor bacterial motility in 3D porous media, 4 mL of the jammed hydrogel media containing bacteria was confined in the bottom of a sealed glass-bottom petri dish (packing thickness ˜1 cm) with an overlying thin layer (750 μl) of LB to prevent evaporation. A Nikon A1R inverted laser-scanning confocal microscope with a temperature-controlled stage at 30° C. was used to capture fluorescence images every 69 ms from an optical slice of 79 μm thickness. The images are captured at least 100 μm from the bottom of the container to avoid any boundary effects. An in-house custom MATLAB script was used to track the individual cells, identifying each cell center using a peak finding function with subpixel precision and tracking the cells using the classic Crocker-Grier algorithm. Between 500 and 1500 cells were tracked for each porous medium tested. The imaging time scale for is shorter than the cell division time, ensuring that measurements of motility are not influenced by cellular growth and division. Over the experimental time scale (˜30 min) in this example, using trapped cells as tracers of matrix deformations, no changes in the pore structure of the packing were detected due to evaporation, swelling, or microbial activity.

This imaging yields a 2D projection of cell motion in 3D; the measurements therefore likely underestimate the cell speeds and hopping lengths, and likely overestimate the trapping durations. However, the measurements of hopping and trapping are robust to variations in the choices of the threshold speed cut off and the minimum tracking duration, suggesting that errors due to projection effects do not play an appreciable role. To further estimate the error due to 2D projection, the polar angle below which any cells moving in 3D will be erroneously identified as trapped are estimated. The threshold speed cut off to define a trapped cell corresponds to a maximum 2D displacement of ˜1 μm per frame; thus, a cell would be erroneously considered to be trapped if it were moving out of the imaging plane at a polar angle smaller than θ*=tan⁻¹(1 μm/39.5 μm) from the vertical axis, where 39.5 μm is half the imaging slice thickness. The corresponding total solid angle is therefore 2×4π sin² θ*, where the factor of two accounts for both upward and downward motion. This solid angle is 0.13% of the total solid angle of the sphere, 4π. Given that the measured velocities are isotropically oriented, this estimate indicates that only 0.13% of cell motions are erroneously characterized due to 2D projection.

To determine the experimental uncertainty in the measured instantaneous speed

$$v ( t ) ≡ \frac{\vert \vec{r} ( t + δ t ) - \vec{r} ( t ) \vert}{δ t} ,$$

the uncertainty in the ability to measure the instantaneous position \vec{r}(t) can be determined by tracking a completely immobile cell trapped in the densest porous medium. The MSD remains constant at 0.017 μm², corresponding to a positional uncertainty of Δr=\sqrt{10.017 μm²}=130 nm. The uncertainty in v is thus

$$\frac{2 Δ r}{δ t} = 4 µm / s .$$

To determine the corresponding uncertainty in the measured velocity reorientation angle, the maximal error in δθ can be calculated for two consecutive velocity vectors \vec{v}(t) and \vec{v}(t+δt) arranged such that δθ=0, i.e., perfectly directed motion. The maximal uncertainty in δθ thus determined is ≈0.25 rads.

To measure the chord length distribution, maximum-intensity time-projections of movies of dispersed 200 nm fluorescent nanoparticles diffusing through the pore space can be constructed. This provides a snapshot of the pore space geometry. These time projections can be binarized into two phases—pore space and solid matrix—and lay randomly oriented lines across each image. A chord is defined as a line segment of length with every point in the pore space. We use these measurements to generate a discrete probability density function for each porous medium from all chord lengths, f(Lₕ).

Measurement of long-time diffusivity; To measure the long-time translational diffusivity of bacteria within a porous medium, a suspension of E. coli was premixed in a jammed medium of hydrogel particles to a final concentration of 8 million cells per μL. A small bolus (˜190 nL) of this mixture was then injected with a micronozzle inside an initially cell-free 0.5% jammed hydrogel porous medium and, after allowing the porous medium to self-heal, was imaged using confocal microscopy. The radial spreading of this population due to cellular motility can be quantified by taking a maximum-intensity time-projection (spanning 10 min) both immediately after injecting the bolus and after, e.g., 103 min. From the azimuthally averaged intensity profiles, the position of the bolus boundary can be determined by defining a threshold intensity and then using that threshold to measure the initial bolus radius Rₒ. The spread bolus radius Rₒ+ΔR can then be measured after a period of time, e.g., 103 minutes in this example. By tracking the bolus boundary, complications due to growth and division of trapped cells in the center of the bolus can be avoided. Given that the spreading is isotropic in an effectively unbounded 3D medium, ΔR can be approximated by the one-dimensional diffusion length, and the diffusivity is therefore given by

$$\frac{{( Δ R )}^{2}}{4 Δ t} = \frac{{( 225 µm )}^{2}}{4 ( 103 min )} = 2 {µm}^{2} / s .$$

The overall change in the amount of nutrient levels is given by ΔC=kC_{b}(V_{B}/V)/Δt, where is the nutrient consumption rate per cell C_{b}, is the bacterial concentration in the bolus used in our diffusivity measurements (corresponding to dilute cell volume fractions less than 0.6 vol % in this example), V_{B}≈190 nL is the bolus volume, V≈25×10³V_{B} is the total volume of the medium, and Δt˜100 min is the experimental time scale; the fractional change in nutrient is thus given by ΔC/C, where C is the initial dissolved nutrient concentration throughout the medium. As a representative example, the consumption of oxygen or essential amino acids for E. coli (e.g. L-serine, L-aspartate) is considered. Using measured values of C and k, fractional changes in nutrient levels smaller than 0.06% can be calculated over the experimental timescale. It is therefore expected that nutrient limitation does not play a role in this example.

It is also expected that the spatial profile of nutrients experienced by single cells is uniform in our experiments. In the diffusion experiments, nutrient consumption by individual cells could generate cell concentration-dependent spatial inhomogeneities throughout the porous medium. For the range of concentrations explored here, inhomogeneities arising from nutrient consumption are rapidly homogenized by nutrient diffusion through the porous media, and thus, we do not expect cell concentration-dependent effects. This result can be seen via a calculation of the competition between nutrient consumption throughout a spherical bolus of cells and diffusion from the bolus boundary. The timescale of nutrient consumption is given by C/kC_{b}, and thus, the length scale over which the nutrient level varies is given by the diffusion length

$$2 \sqrt{D ( \frac{C}{k {C}_{b}} )} ,$$

where D is the diffusivity of single nutrient molecules. Because the hydrogel polymer is less than 1% of the total mass of the system, and the mesh size is ˜100 nm (much larger than ˜nm-sized single molecules), it can be assumed that the hydrogel does not alter nutrient transport and availability. Using measured values of D, diffusion lengths of 2.0, 5.8, and 1.2 mm can be calculated—three orders of magnitude larger than the size of a single bacterium—for the three representative examples of oxygen, L-serine, and L-aspartate. It is therefore expected that the spatial profile of nutrients experienced by single cells is uniform in these experiments.

This experiment was repeated four more times, testing bacterial concentrations of either 4-8 million cells per μL or 40-60 thousand cells per μL, and testing three different characteristic pore sizes α=3.6, 2.5, 1.9 μm. Importantly, the measurements performed on populations of different concentrations within media of the same pore size yield comparable values of diffusivity, indicating that the measurements are independent of cell concentration and are indeed representative of non-interacting single cells.

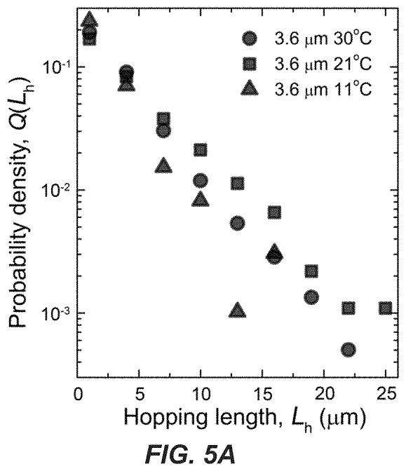

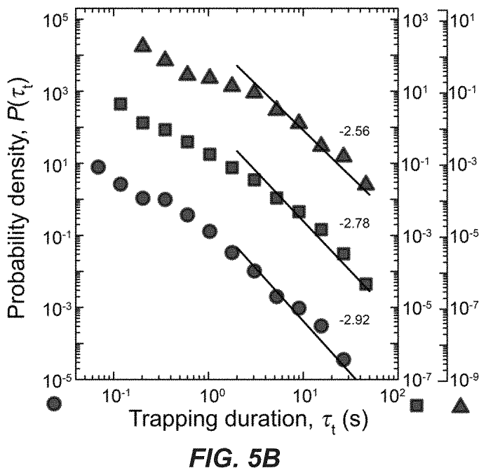

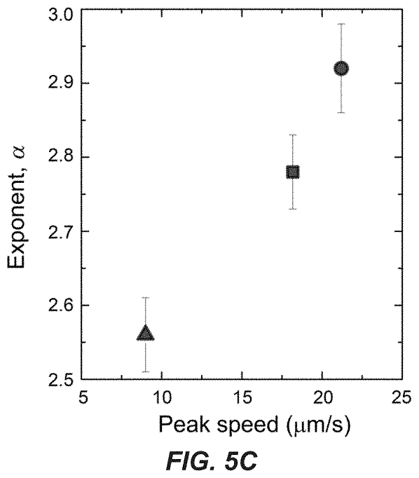

In some embodiments, changes in growth/motility due to changes in temperature can be quantified. In one example, identical systems with characteristic pore sizes of 3.6 μm at 30° C. (dark circles in FIGS. 5A-5C), 21° C. (dark squares in FIGS. 5A-5C), and 11° C. (dark triangles in FIGS. 5A-5C) are monitored. As seen in FIG. 5A, the probability densities for different hop lengths are very similar, with the 21° C. tending to have slightly higher probability densities for a given hop length, and the 11° C. tending to have slightly lower probability densities. However, there was no systematic variation of Q(Lₕ) with temperature. As seen in FIG. 5B, the probability distributions for different trapping durations are also very similar. Broad distributions of τₜ were observed, with extended tails consistent with power-law scaling for large τ₆>τ₀≈2 s in all cases: P(τₜ)˜τₜ^{-α} as shown by the lines in FIG. 5B, where the scaling is −2.56 for the 11° C. media, −2.78 for the 21° C. media, and −2.92 for the 30° C. media. The power-law exponent determined from the complementary cumulative distribution function decreases with decreasing cellular activity, consistent with a prediction that α=1+X/C₀; this behavior is summarized in FIG. 5C, where the exponent is shown for the peak speeds of the bacteria in the different temperature media.

In various embodiments, the porous medium can be used for antibiotic assays and/or high throughput screenings of, e.g., antibiotics or other compounds.

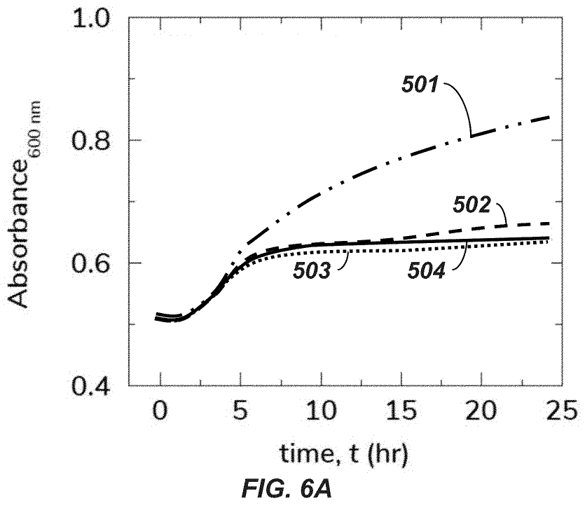

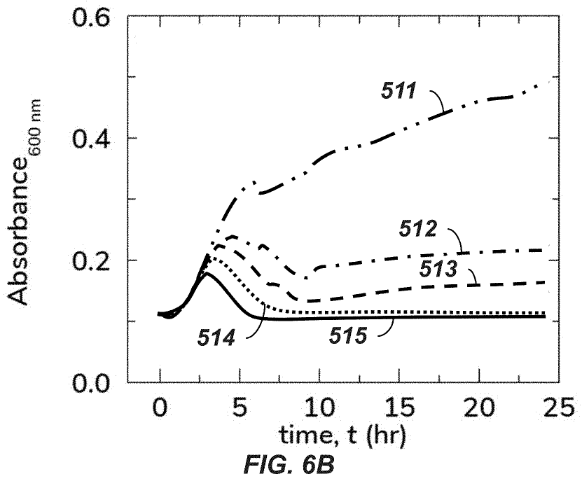

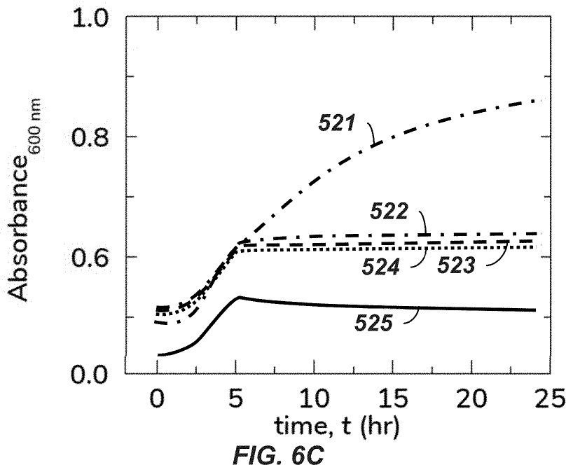

In some embodiments, changes in growth/motility due to changes in antibiotic or antibiotic concentrations can be quantified. For example, FIG. 6A shows growth curve measurements for different concentrations of kanamycin in different areas of the same porous media, with similar characteristic pore sizes. Using an 0.81% gel with different concentrations of kanamycin (675 μg/ml (ref 504), 317.5 μg/ml (ref 503), 156.25 μg/ml (ref 502), and 78.125 μg/ml (ref 501)), it can be seen that for the two highest concentrations, there was no growth. For the 156.25 μg/ml (ref 502) of kanamycin, only a small amount of growth was seen, but a relatively high amount of growth was seen at 78.125 μg/ml (ref 501). Concentrations less than 78.125 μg/ml performed similarly to the 78.125 μg/ml concentration.

The efficacy of antibiotics in porous versus non-porous media can also be quantified, as seen in FIGS. 6B and 6C. In both figures, different concentrations of ampicillin were tested 1 μg/ml (ref. 511, 521), 2 μg/ml (ref 512, 522), 3 μg/ml (ref 513, 523), 4 μg/ml (ref 514, 524) and 6 μg/ml (ref 515, 525). For FIG. 6B, the different concentrations were added to bacteria in liquid LB media with no shaking. As can be seen, no growth was seen in the 4 and 6 μg/ml concentrations, while growth was detected in the other concentrations. For FIG. 6C, however, the different concentrations were added to porous media with similar pore characteristics, and as can be seen, only the 1 μg/ml concentration showed growth.

In FIGS. 6A-6C, the levels of bacteria were monitored by measuring light absorbance at 600 nm. In some embodiments, the cells or media is irradiated with light from a light source positioned above the cells, and a lens or sensor positioned below the cells then captures the light either being emitted from the cells (i.e., the intensity of light being emitted by a marker gene, etc.) or being transmitted through the cells (i.e., not being absorbed). A processor can then be used to quantify the measured light or sensor signals. Further a processor can also be used to convert the measured light or sensor signals into concentrations or bacterial cell counts, via, e.g., calibration curves. Based on that, various antibiotic performance characteristics can be determined—MIC, minimal bactericidal concentration (MBC), etc.

In some embodiments of a high-throughput screen, the porous medium replaces a traditional multi-well plate or array, and instead different areas within the medium are used to contain the different screening compounds. In other embodiment of a high-throughput screen, after a porous media is created with a substantially uniform dispersion of the bacterial cells (for example, every cubic centimeter of the porous medium contains the same amount of bacterial cells ±10%), portions of the porous medium are introduced into different wells in a multi-well plate of array, ensuring substantially identical starting compositions.

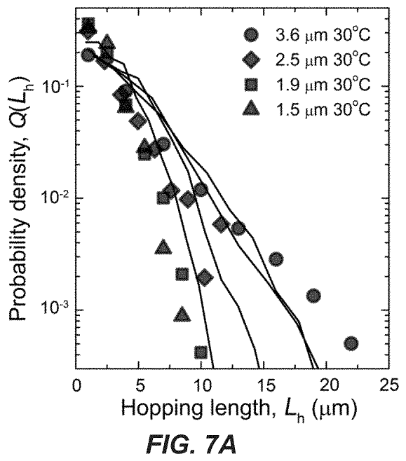

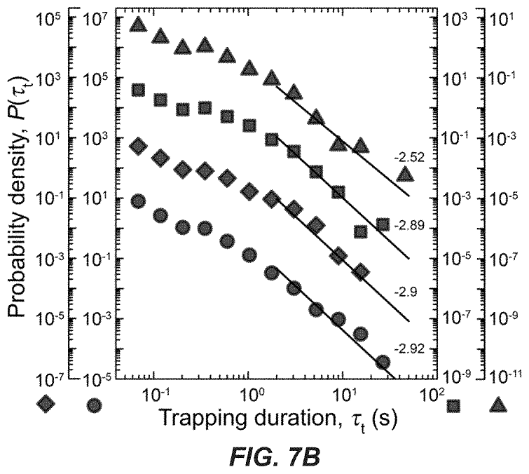

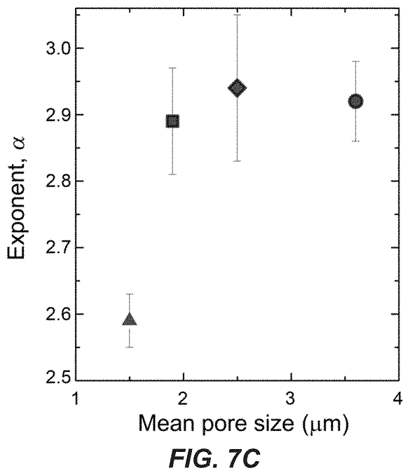

In some embodiments, changes in cell confinement based on pore size can be quantified. In one example, systems with different characteristic pore sizes—1.5 μm (dark triangles in FIGS. 7A-7C), 1.9 μm (dark squares in FIGS. 7A-7C), 2.5 μm (dark diamonds in FIGS. 7A-7C), and 3.6 μm (dark circles in FIGS. 7A-7C)—all at the same temperature of 30° C. As seen in FIG. 7A, the probability densities Q(Lₕ) for different hop lengths vary significantly based on pore size. Further, the solid lines indicate the measured chord length distribution Ξ(Lₕ) for each medium, and they also vary significantly based on pore size. Both Q(Lₕ) and Ξ(Lₕ) narrow as the characteristic pore size is decreased, and reasonable agreement is found between the two. As seen in FIG. 7B, the probability densities for different trapping durations are similar. As with variations in temperature, broad distributions of τₜ were observed, with extended tails consistent with power-law scaling for large τₜ>τ₀≈2 s in all cases: P(τₜ)˜τₜ^{-α} as shown by the lines in FIG. 5B, where the scaling is −2.52 for the 1.5 μm media, −2.89 for the 1.9 μm media, −2.9 for the 2.5 μm media, and −2.92 for the 3.6 μm media. The power-law exponent determined from the complementary cumulative distribution function decreases with decreasing characteristic pore size, consistent with a prediction that α=1+X/C₀; this behavior is summarized in FIG. 7C, where the exponent is shown for the peak speeds of the bacteria in the media with different characteristic pore sizes. Intriguingly, a appears to decrease more precipitously as pore size decreases below the cell body length≈2 μm, indicating that confinement plays a more dominant role in this regime.

Those skilled in the art will recognize or be able to ascertain using no more than routine experimentation, many equivalents to the specific embodiments of the invention described herein. Such equivalents are intended to be encompassed by the following claims.

What is claimed:

1. A 3D porous medium, comprising:
(a) a self-healing jammed support structure comprising a plurality of transparent hydrogel particles or emulsion droplets;
(b) a bacterial nutrient of a desired composition in the open volumes between transparent hydrogel particles or emulsion droplets and within micropores in the transparent hydrogel particles; and
(c) a plurality of bacterial cells within at least some of the open volume formed between transparent hydrogel particles.

2. The 3D porous medium according to claim 1, further comprising at least one polymer.

3. The 3D porous medium according to claim 1, further comprising a test mineral or chemical in the open volumes between transparent hydrogel particles, within micropores in the transparent hydrogel particles, or a combination thereof.

4. The 3D porous medium according to claim 1, wherein a composition of the bacterial nutrient in a first portion of the self-healing jammed support structure is different from a composition of the bacterial nutrient in a second portion of the self-healing jammed support structure.

5. The 3D porous medium according to claim 1, wherein the mass fraction of the plurality of transparent hydrogel particles is between 0.5%-2.5% of the combined mass of the plurality of transparent hydrogel particles and the bacterial nutrient.

6. The 3D porous medium according to claim 1, wherein the pH of the at least one bacterial nutrient is less than about 7.4

7. The 3D porous medium according to claim 1, wherein the plurality of bacterial cells comprises a plurality of strains of bacteria, each strain present at a different location within the porous medium.

8. The 3D porous medium according to claim 1, wherein each of the plurality of bacterial cells comprises one or more marker genes.

9. The 3D porous medium according to claim 1, wherein an internal mesh size of the self-healing jammed support structure is between 5 nm and 200 nm.

10. The 3D porous medium according to claim 1, wherein each of the plurality of hydrogel particles has a diameter between 10 nm and 100 μm.

11. The 3D porous medium according to claim 1, wherein the 3D porous medium consists essentially of the plurality of transparent hydrogel particles, the plurality of bacterial cells, and the bacterial nutrient.

12. A kit for creating 3D porous media, comprising:
(a) hydrogel particles; and
(b) a liquid or powder capable of being used by itself, or with the addition of DI water, to form a bacterial growth medium adapted for swelling the hydrogel particles.

13. A method for producing a porous medium incorporating bacteria, comprising:
providing a porous medium;
using a nozzle to introduce a bacterial cell suspension to at least one location within the porous medium, where each bacterial cell in the bacterial cell suspension comprises a marker gene;
removing the nozzle; and
allowing the porous medium to at least partially self-heal.

14. The method according to claim 12, further comprising continuously introducing the bacterial cell suspension into the porous medium as the nozzle is moved within the porous medium from a first location to a second location.

15. The method according to claim 12, further comprising allowing the bacterial cells to grow and move within the porous medium.

16. The method according to claim 12, further comprising utilizing fluorescence microscopy to visualize cell fluorescence.

17. The method according to claim 12, further comprising interacting with a bacterial cell in the porous medium.

18. The method according to claim 12, wherein providing a porous medium comprises swelling dry hydrogel granules in a bacterial cell culture media at a predetermined concentration.

19. The method according to claim 12, wherein at least one attribute of the porous medium is controlled using different hydrogel concentrations, wherein the attribute is selected from the group consisting of pore size, permeability, porosity, elastic modulus, viscous modulus, and yield stress.

20. The method according to claim 12, further comprising modifying the porous medium by adding a test mineral or chemical to at least one location within the porous medium.

21. The method according to claim 12, further comprising controlling fluid flow, nutrient signals, chemical signals, or a combination thereof throughout the porous medium using microfluidic channels.

22. The method according to claim 12, further comprising characterizing microbial motility, microbial growth, or both.
